(19) United States
(12) Patent Application Publication
(10) Pub. No.: US 2009/0061022 A1
(43) Pub. Date: Mar. 5, 2009

(54) Pharmaceutical composition comprising arsenite for the treatment of malignancy

(76) Inventor: Sang Bong Lee, Anyang (KR)

Correspondence Address: Sang Bong Lee;(Attn: Je Ok LEE), 2746 W. 235th. St., #9, Torrance, CA, 90505, US

(21) Appl. No.: 12/229,264

(22) Filed: Aug. 21, 2008

Related U.S. Application Data
(63) Continuation-in-part of application No. 10/962,357, filed on Oct. 8, 2004.

(51) Int. Cl. A61K 33/36; A61P 35/00; A61P 35/02

(52) U.S. Cl. 424/623

(57) ABSTRACT

A method for using sodium meta-arsenite (AsO₂⁻) to suppress growing of human cancer cells is provided to find a method of curing human cancers. Effects of sodium-meta arsenite (AsO₂⁻) on various human cancer cells are investigated via in vitro cytotoxic activity and in vivo anticancer activity against nude mice. For in vitro cytotoxic activity, six (6) kinds of human cancer with nine (9) cancer cell lines, eight (8) kinds of human cancers with twelve (12) cancer cell lines and ten (10) kinds of human cancers with forty one (41) cancer cell lines are tested. For in vivo anticancer activity, two (2) kinds of cancers of human renal cancer RXF 944LX and leukemia cells are tested utilizing nude mice. Maximum tolerated dose assessment of sodium meta-arsenite (AsO₂⁻) after daily oral gavages for 14 days were investigated for mouse, rat and pig. Subacute toxicity study of sodium meta-arsenite (AsO₂⁻) was induced after daily oral gavages for 14 days in the mouse followed by a 28-day recovery period. Arsenic acid sodium salt and cacodylic acid were used for comparison of the effect on the same human cancer cell lines. Sodium meta-arsenite (AsO₂⁻) showed better anticancer activity without any severe side effects within the range of tested dose compared to other arsenic compounds.

Current application is a continuation-in-part of the U.S. patent application Ser. No. 10/962,357, which is now abandoned. The present application relates to a method of suppressing human cancer cells to find a curing method of various human cancers, especially with sodium meta-arsenite (AsO₂⁻).

### FIELD OF INVENTION

### Background of Invention

Potassium arsenite solution had been used in the treatment of leukemia, particularly in chronic myeloid leukemia (CML), erythemia, and Hodgkin's lymphoma since 1865 in the form of Fowler's solution. But, that was abandoned due to chronic toxicity with long-term use. Two arsenic compounds have been utilized in Chinese traditional medicine for more than 500 years. One is white arsenic, essentially containing arsenic trioxide (As₂O₃), which was recorded in the Compendium of Material Medica (edited by Li Shizhen, 1518-1593) and still used clinically in the treatment of certain skin diseases and asthma and to promote the healing of surgical wounds. The other compound is realgar compound, which contains arsenic sulfide and has been administered in the treatment of CML for more than 40 years. In 2000, FDA approved use of Arsenic trioxide (As₂O₃) as therapeutic agent of the acute promyelocytic leukaemia (APL). However, arsenic trioxide is extremely poisonous, sparingly and extremely slowly soluble in cold water. Arsenic trioxide has LD₅₀ in rats 15.1 mg/kg orally (Merck Index, 2001). It is the purpose of the current application to find a less toxic arsenic compound and to find a method of using it as a human anticancer drug.

### SUMMARY OF THE INVENTION

Potassium arsenite solution had been used in the treatment of leukemia, erythemia, and Hodgkin's lymphoma since 1865 in the form of fowler's solution. But, that was abandoned due to chronic toxicity with long-term use. Two arsenic compounds have been utilized in Chinese traditional medicine. One is white arsenic, essentially containing arsenic trioxide (As₂O₃), which still used clinically. The other compound is realgar compound, which contains arsenic sulfide and has been administered in the treatment of CML for more than 40 years. FDA approved use of arsenic trioxide (As₂O₃) as a therapeutic agent of the APL in 2000. However, arsenic trioxide is extremely poisonous. It is the purpose of the current application to find a less toxic arsenic compound and find a method of using it as a human anticancer drug. Effects of sodium-meta arsenite (AsO₂⁻) on various human cancer cells are investigated via in vitro cytotoxocity and in vivo anticancer activity against nude mice. For in vitro cytotoxic activity, six (6) kinds of cancers with nine (9) cancer cell lines, eight (8) kinds of cancers with twelve (12) cancer cell lines and ten (10) kinds of cancers with of forty one (41) cancer cell lines are tested. For in vivo anticancer activity, two (2) kinds of cancer cell lines of human renal cancer RXF 944LX and leukemia are tested utilizing nude mice. Maximum tolerated dose assessment of sodium meta-arsenite (AsO₂⁻) after daily oral gavages for 14 days were investigated for mouse, rat and pig. Subacute toxicity study of sodium meta-arsenite (AsO₂⁻) was induced after daily oral gavages for 14 days in the mouse followed by a 28-day recovery period. Arsenic acid sodium salt and cacodylic acid were used for comparison of the effect on the same human cancer cell lines. Sodium meta-arsenite (AsO₂⁻) showed better anticancer activity without any severe side effects within the range of tested dose compared to other arsenic compounds.

### BRIEF DESCRIPTION OF DRAWINGS

FIG. 1 is an IC₇₀ profile of arsenic acid sodium salt in a panel of 12-human cancer cell lines.

FIG. 2 is an IC₇₀ profile of sodium meta-arsenite in a panel of 12-human cancer cell lines.

FIG. 3 is an IC₇₀ profile of arsenic trioxide in a panel of 41-human cancer cell lines.

FIG. 4 is an IC₇₀ profile of sodium meta-arsenite in a panel of 41-human cancer cell lines.

### DETAILED DESCRIPTION OF THE PREFERRED EMBODIMENT

### Experiments

To investigate the possibility of sodium meta-arsenite as a human anticancer drug, various kinds of experiments required by FDA or EORTC or other country's guidelines for approval for new anticancer drugs are induced. Effects of sodium-meta arsenite on various human cancer cells are investigated by in vitro cytotoxic activity and in vivo anticancer activity against nude mice. For in vitro cytotoxic activity, six (6) kinds of human cancers with nine (9) cancer cell lines, eight (8) kinds of human cancers with twelve (12) cancer cell lines and ten (10) kinds of human cancers with of forty one (41) cancer cell lines are tested. For in vivo anticancer activity, two (2) kinds of human renal cancer cell line RXF 944LX and leukemia are tested utilizing nude mice. Maximum tolerated dose assessment of sodium meta-arsenite after daily oral gavages for 14 days were investigated for mouse, rat and pig. Subacute toxicity study of sodium meta-arsenite was induced after daily oral gavages for 14 days in the mouse followed by a 28-day recovery period.

### 1. In Vitro Cytotoxic Activity

### 1.1 Cytotoxic Activity Against Human Cancer Cell Lines

The cytotoxic activity of sodium meta-arsenite and two other arsenic compounds (arsenic acid sodium salt and cacodylic acid) were initially analyzed in in vitro against a panel, which contains different nine human cancer cell lines that are derived from human leukemia and solid cancers and followed by continuous drug exposure for 3-7 days. Same tests were induced and analyzed for twelve (12) and forty-one (41) human cancer cell lines.

1-1-A. Stock solution Preparation

Three kinds of stock solutions were prepared for in vitro cytotoxic activity test. The first solution is DMSO (dimethyl sulfoxide) mixed with sodium meta-arsenite. The second solution is DMSO mixed with cacodylic acid (dimethylarsinic acid). The third solution is DMSO mixed with arsenic acid sodium salt. Those three solutions were diluted with culture medium (RPMI 1640). The final concentrations of DMSO in those three solutions are less than 0.3 vol %.

### 1-1-B. Cancer Cell Sample Preparation

Nine kinds of human cancer cell lines out of six kinds of cancers were grown at 37° C. under a controlled atmosphere of 95% air and 5% CO₂. The cells were grown on monolayer cultures of RPMI 1640 cell culture medium supplemented with 10% fetal calf serum.

The grown cells were harvested from exponential phase cultures by trypsinization and counted. The counted cells were placed in a plate, which has ninety six wells. Each well on the plate is in a shape of a flat-bottomed microtitre. Density of the cells in a suspension was adjusted depending on the cell line. The suspension was prepared by diluting the grown cells with the culture medium (RPMI 1640). Concentration of the cells were varied from 5×10⁴ to 1×10⁵ viable cells/ml. 100 μl of the suspension was placed in each well of the plate. After 24-hours, the cells start to recover. Then add 50 μl of culture medium (RPMI 1640) only to 6 wells, 50 μl/well, among the 96 wells of one plate. It is called controlled. One well is filled with culture medium plus 5-FU. And another 50 μl of culture medium plus one of the stock solutions were added to each of the left 89 wells to allow cells to resume exponential growth. The concentration of the stock solutions were varied as 1 μg/ml, 3 μg/ml, 10 μg/ml, 30 μg/ml, and 100 μg/ml to find out the cytotoxic activity of each Arsenic compound.

### 1-1-C. Cytotoxic Activity Against Nine (9) Human Cancer Cell Lines

The cytotoxic activity test was executed with three plates simultaneously to check reliability of the data obtained. The three plates contain the same samples of cancer cells. The three plates were then incubated for 3-7 days under the same controlled atmosphere and cell proliferation was assessed with a modified propodium iodide fluorescence assay. After thawing of the plates, fluorescence was measured using a Millipore Cytofluor 2350-microplate reader (excitation 530 nm emission 620 nm) in order to quantify the total cell number. The assay included untreated and positive controls containing 5-FU.

IC₅₀, IC₇₀ and IC₉₀ values were determined as the drug concentration required to reduce cell growth down to 50, 70, and 90% of the growth rate of the untreated control (the six well samples).

Table 1 shows the results of the experiments for sodium meta-arsenite in the panel of six human cancer cell lines from solid cancers listed and three kinds of leukemia (one cell line is from lymphoma).

Growth inhibition is defined as (numbers of cancer cell alive) treated (T)/(number of cells) control (C)×100% (T/C %). Anticancer activity is defined as growth inhibition of cancer cell lower than 30% of the medium (RPMI 1640) only treated controlled cells (T/C<30). Sodium meta-arsenite was very active to lymphoma cell line when the concentration of sodium meta-arsenite in the stock solution is at 1 μg/ml. When the concentration of sodium meta-arsenite increased as 3 μg/ml, sodium meta-arsenite showed activity on two (2) out of eight (8) cancer cell lines, leukemia K562, lymphoma U937, and 25%.

At a concentration of 10 μg/ml, sodium meta-arsenite showed activity on three (3) out of nine (9) cancer cell lines, leukemia K562, lymphoma U937, LXFL 529L, and 33%.

At a concentration of 30 μg/ml, sodium meta-arsenite showed activity on four (4) out of eight (8) cancer cell lines, leukemia K562, lymphoma U937, colon cancer HT29, LXFL 529L, 50%.

At a concentration of 100 μg/ml, sodium meta-arsenite showed activity on eight (8) out of nine (9) cancer cell lines and the most sensitive cancer cell line against sodium meta-arsenite was lymphoma U937. The order of sensitivity to sodium meta-arsenite is in the order of U937 of lymphoma>LXFL 529L of lung cancer>RXF944L of renal cancer>K562 of leukemia>HT 29 of colon cancer.

TABLE 1
Cytotoxic activity of sodium meta-arsenite
in a panel of 9 human cancer cell lines

| | T/C % at drug concentration (μg/ml) | | | | |
|---|---|---|---|---|---|
| | Human cancer cell Lines | | | | |
| | 1 | 3 | 10 | 30 | 100 |
| Colon Cancer | | | | | |
| HT29 | 91 | 60 | 40 | 22 | 14 |
| Leukemia | | | | | |
| CCRF-CEM | 48 | | 43 | | 29 |
| K562 | 51 | 15 | 15 | 18 | 13 |
| U937 | 3 | 3 | 2 | 2 | 1 |
| NSCLC | | | | | |
| LXFL 529L | 79 | 61 | 21 | 7 | 4 |
| Melanoma | | | | | |
| MEXF 462NL | 112 | 120 | 101 | 91 | 15 |
| MEXF 514L | 120 | 95 | 98 | 70 | 21 |
| Renal cancer | | | | | |
| RXF 944L | 124 | 79 | 57 | 32 | 9 |
| Uterine body | | | | | |
| UXF 1138 | 107 | 114 | 103 | 95 | 30 |
| Active/Total | 1/9 | 2/8 | 3/9 | 4/8 | 8/9 |
| cancer cell lines | 11% | 25% | 33% | 50% | 89% |

The difference of three arsenic compounds in activity against various cancer cell lines are summarized as their mean IC₅₀, IC₇₀ and IC₉₀ values shown in Table 2. Sodium meta-arsenite is the most potent. The dose-response curve of sodium meta-arsenite was rather steep and those of cacodylic acid and arsenic sodium salt were more flat.

Comparison of the IC₅₀ values showed that sodium meta-arsenite was the most potent compound of the series, followed by arsenic acid sodium salt and cacodylic acid. The latter compounds should be considered as inactive.

TABLE 2
Mean IC₅₀, IC₇₀ and IC₉₀ values of three different arsenic
compounds against various human cancer cell lines tested

| | IC₅₀ μg/ | IC₇₀ μg/ | IC₉₀ μg/ |
|---|---|---|---|
| Compound | ml (μM) | ml (μM) | ml (μM) |
| Sodium meta-arsenite | 7.3 (56) | 19.8 (152) | 63.7 (490) |
| Arsenic acid sodium salt | 30.8 (99) | 44.7 (143) | 77.5 (248) |
| Cacodylic acid | 96.1 (696) | 98.4 (713) | >100 (>725) |

### 1-1-D. Cytotoxic Activity Against Twelve (12) Human Cancer Cell Lines

In a further study, the cytotoxic activity profile of sodium meta-arsenite and arsenic acid sodium salt was investigated in different panels containing twelve (12) human cancer cell lines from eight (8) kinds of human cancers. The cancer cell samples were prepared as described in 1-1-B. Propidium iodide fluorescence assay method for this test was modified as the follows.

A stock solution of sodium meta-arsenite was prepared in PBS (Phosphate Buffered Saline) (33 mg/ml) and further diluted with culture medium as appropriate;
1) cell density ranged 5,000-12,000 viable cells/well,
2) test compound was applied at 5 doses with one log increments ranging from 0.001 to 10 μg/ml,
3) culturing time was 4 days.

TABLE 3
Mean IC₅₀, IC₇₀, and IC₉₀ values of sodium meta-arsenite and arsenic
acid sodium salt in a panel of 12 human cancer cell lines

| | IC₅₀ μg/ | IC₇₀ μg/ | IC₉₀ μg/ |
|---|---|---|---|
| Compound | ml (μM) | ml (μM) | ml (μM) |
| Sodium meta-arsenite | 0.9 (7) | 2.0 (15) | 6.8 (52) |
| Arsenic acid sodium salt | 25.5 (82) | 40.1 (129) | 76.6 (246) |

Table 3 shows that sodium meta-arsenite was over 20 times more potent than arsenic acid sodium salt in μg/ml, which approximately 10 times if counted on a molar basis.

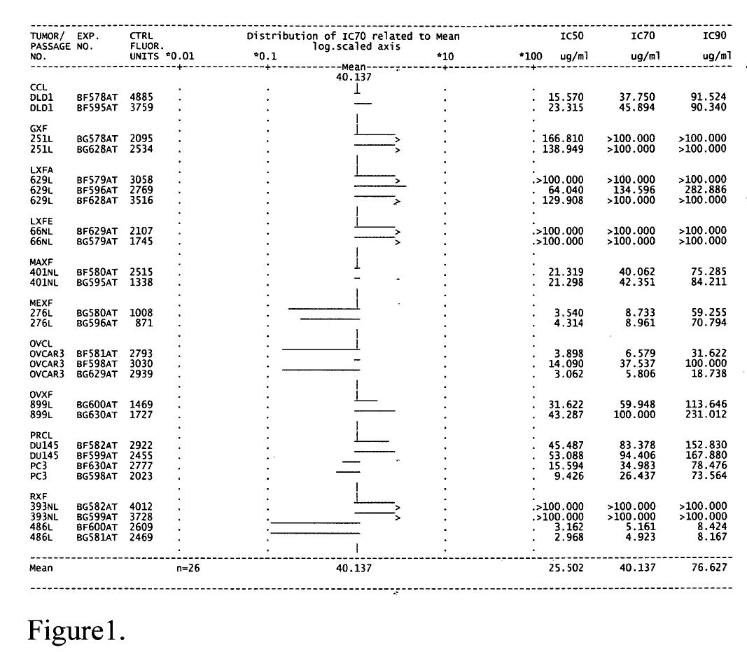

However, the cytotoxic activity profiles summarized in FIGS. 1 & 2 show different results.

In the FIG. 1, abbreviations are as follows; CCL-colon cancer cell line, GXF-gastric cancer cell line (xenograft), LXFA-lung cancer (adenocarcinoma) cell line (xenograft), LXFE-lung cancer (epidermoid) cell line (xenograft), MAXF-mammary cancer cell line (xenograft), MEXF-melanoma cell line (xenograft), OVCL-ovarian cancer cell line, OVXF-ovarian cancer cell line (xenograft), PRCL-prostate cancer cell line, RXF-renal cancer cell line (xenograft).

The IC₇₀ profile of arsenic acid sodium salt, shown in FIG. 1, revealed that the renal cancer cell line (xenograft) RXF 486L, melanoma cancer cell line (xenograft) MEXF 276L and ovarian cell line OVCAR3 were the most sensitive cancer cell lines to the arsenic acid sodium salt. The cell lines of GXF 251L (gastric), LXFA 629L and LXFE 66NL (non-small cell lung) and RXF393NL (renal) were resistant (IC₇₀>100 μg/ml) to the arsenic acid sodium salt.

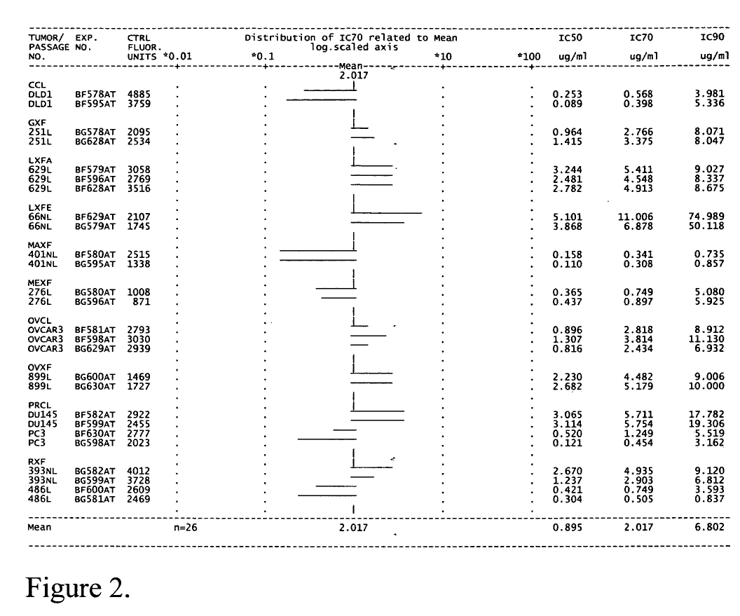

The IC₇₀ profile of sodium meta-arsenite, shown in FIG. 2, is also different from that of arsenic acid sodium salt. In the FIG. 2, bars extending to the left represent sensitivity of the cell line to sodium meta-arsenite in access of the average sensitivity of all tested cell lines. Bars extending to the right correspondingly imply sensitivity less than the mean.

Sodium meta-arsenite was active in all 12 human cancer cell lines (100%) at the dose levels of 100 μl/ml and 10 μg/ml. But, at 1 μg/ml, sodium meta-arsenite was active in four (4) out of twelve (12) human cancer cell lines (33%). At a dose level lower than 1 μg/ml, the sodium meta-arsenite did not display any cytotoxic activity at all.

In the FIG. 2, the abbreviations are same as the FIG. 1. The most sensitive cell line was MAXF 401NL (mammary), followed by DLD1 (colon), RXF 486L (renal), MEXF 276L (melanoma), and PC3 (prostate).

When the concentration of the sodium meta-arsenite was greater than 100 μg/ml resistance (IC₇₀>100 μl/ml), no resistant to sodium meta-arsenite was observed.

### 1-1-E Cytotoxic Activity Against Forty One (41) Human Cancer Cell Lines

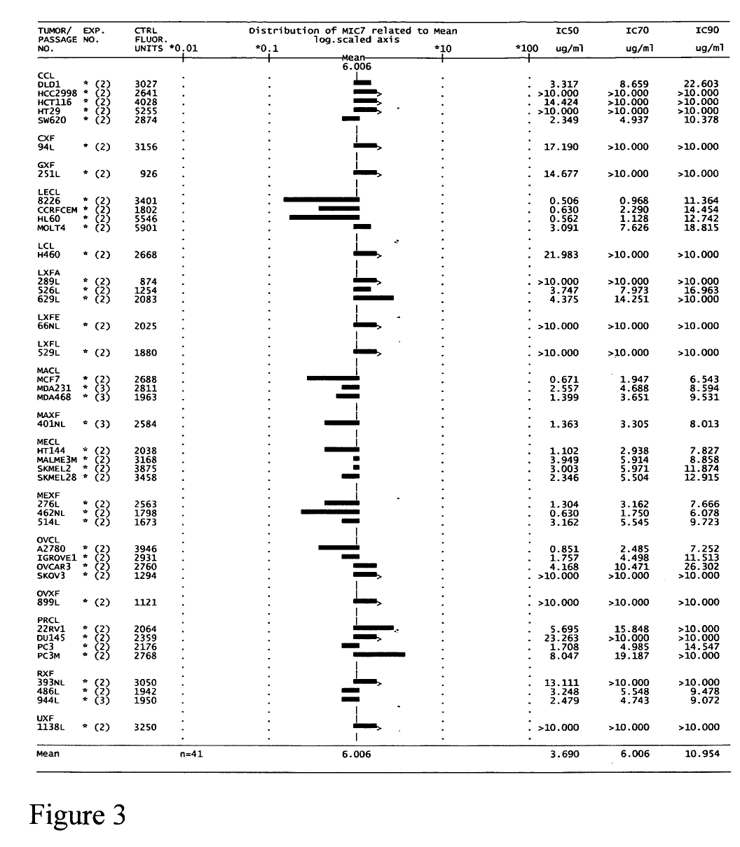

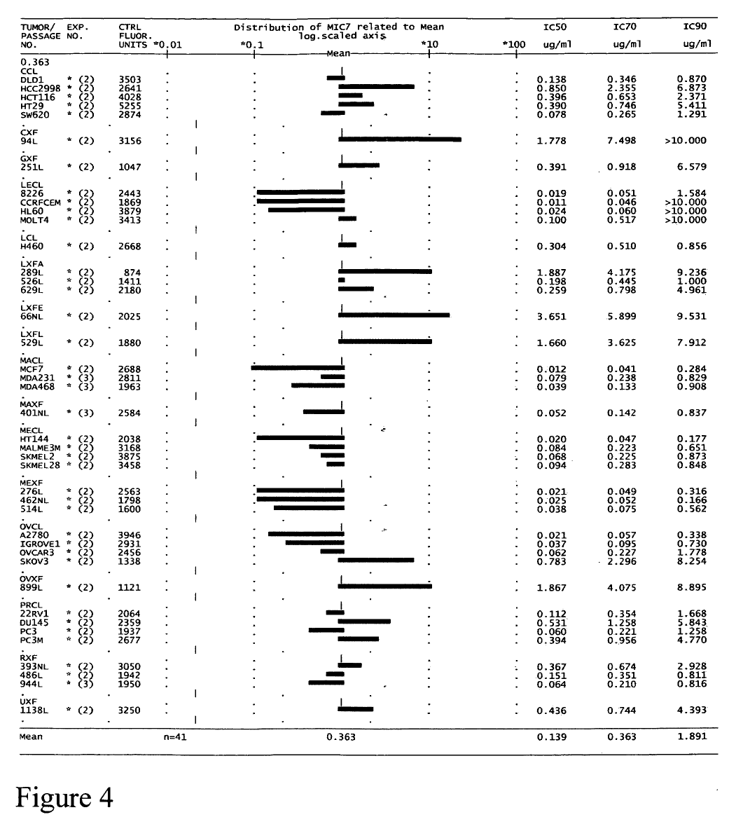

In a next study, the cytotoxic activity of sodium meta-arsenite was head-to-head compared with arsenic trioxide in panels loaded with 41 cancer cell lines from ten human cancers. Experimental condition for set up and cell culture were the same as describe above. The study confirmed the potency and differences in activity of sodium meta-arsenite, compared to other components, shown in the previous study. It also showed that sodium meta-arsenite had another profile and was more potent than arsenic trioxide (FIGS. 3 and 4).

In the FIG. 3 and FIG. 4, the abbreviations are as follows; CCL-colon cancer cell line, CXF-colon cancer cell line (xenograft), GXF-gastric cancer cell line (xenograft), LECL-leukemia cell line, LXFA-lung cancer (adenocarcinoma) cell line (xenograft), LXFE-lung cancer (epidermoid) cell line (xenograft), LXFL-lung cancer (large) cell line (xenograft), MACL-mammary cancer cell line, MAXF-mammary cancer cell line (xenograft), MECL-melanoma cell line, MEXF-melanoma cell line (xenograft), OVCL-ovarian cancer cell line, OVXF-ovarian cancer cell line (xenograft), PRCL-prostate cancer cell line, RXF-renal cancer cell line (xenograft), UXF-uterine body cancer cell line (xenograft).

The mean IC₅₀ and mean IC₇₀ values of sodium meta-arsenite were 0.129 μg/ml (0.99 μM) and 0.329 μg/ml (2.53 μM), respectively. The IC₅₀ and IC₇₀ values of arsenic trioxide were 3.462 μg/ml (17.5 μM) and 6.022 μg/ml (30.44 μM), respectively, which are 27- and 18-fold less potent compared to sodium meta-arsenite, or 17- and 12-fold on the basis of μM shown in Table 4.

TABLE 4
Mean IC₅₀, IC₇₀, and IC₉₀ values of sodium meta-arsenite and arsenic
trioxide in a panel of forty-one (41) human cancer cell lines

| | IC₅₀ μg/ | IC₇₀ μg/ | IC₉₀ μg/ |
|---|---|---|---|
| Compound | ml (μM) | ml (μM) | ml (μM) |
| Sodium meta-arsenite | 0.129 (0.99) | 0.329 (2.53) | 1.691 (13.02) |
| Arsenic trioxide | 3.462 (17.50) | 6.022 (30.44) | 10.307 (52.11) |

The cytotoxic activity of both compounds was dose-dependent.

Sodium meta-arsenite was active in all forty-one (41) cancer cell lines (100%) at the dose of 10 μg/ml. At a dose of 1 μg/ml thirty three (33) out of forty one (41) cancer cell lines (80%) responded to exposure to the sodium meta-arsenite. At a dose of 0.1 μg/ml ten (16) out of forty one (41) cancer cell lines responded. At lower doses of 0.01 μg/ml and 0.001 μg/ml, the sodium meta-arsenite showed no activity.

Arsenic trioxide was only active in 23/41 cancer cell lines (56%) at the dose of 10 μg/ml.

At 1 μg/ml of dose, only one (1) out of forty one (41) cancer cell lines (2%) responded to exposure to arsenic trioxide with more than 70% growth inhibition. There was no observed activity in any of investigated cell lines at three lower dose levels of 0.1 μg/ml, 0.01 μg/ml and 0.001 μg/ml.

The IC₇₀ profile of sodium meta-arsenite demonstrated the pronounced selectivity (individual IC₇₀<½ mean IC₇₀) of ¾ in leukemia, ¾ in breast cancer and 4/7 in melanoma cell lines. Less cytotoxic activity to sodium meta-arsenite was observed in cancer cell lines derived from colon cancer and lung cancer.

The IC₇₀ profile of arsenic trioxide differed from that of sodium meta-arsenite. In contrast to the profile of sodium meta-arsenite, the differential activity was not as pronounced. Nevertheless, the same cancer types were sensitive to arsenic trioxide as for sodium meta-arsenite, i.e. leukemia (¾) mammary (¼) and melanoma ( 2/7). The cancer cell lines that were most sensitive to arsenic trioxide were nearly the same as for sodium meta-arsenite. Cancer types that generally responded less sensitive to arsenic trioxide were colon, lung and prostate cancer cell lines.

In conclusion, the in vitro data demonstrate that sodium meta-arsenite has a good cytotoxic activity against human cancer cell lines with IC₅₀ values in the low 1M range. Furthermore, differential activity was observed in cancer cell lines derived from leukemia, mammary cancer and melanoma.

Sodium meta-arsenite was at least 10-fold more potent than the clinically used agent, arsenic trioxide, and had also a better differential activity.

### 2. In Vivo Anticancer Activity

All animal experimentation was performed under a project license issued by the local authorities in Freiburg or Berlin, Germany. According to the Animal Experimentation Rules, animals were killed before the cancers reached mean diameters of approximately 16 mm.

### 2.1 ‘Dose Finding’ Study of Arsenic Compounds Using Human Renal Cancer Model RXF 944LX In Vivo

In a dose finding study, the potential anticancer activity of sodium meta-arsenite and arsenic acid sodium salt versus arsenic trioxide was examined in the human renal cancer model, RXF 944LX. This cancer was selected because it was sensitive in vitro against all three arsenic compounds.

### 2-1-A. Cancer Cell Preparation

Cancer fragments (1-2 mm in diameter), obtained from xenografts in serial passage in nude mice, were implanted subcutaneously (S.C.) in a flank of male NRMI nude mice (one fragment per mouse). Mice with appropriate cancer volumes (5-7 mm in diameter) were randomly distributed to treatment and control groups (4 mice/group). The start of treatment was day 0. Cancer volume and body weights were measured at least twice per week.

### 2-1-B. Administration

1) Sodium meta-arsenite (3, 10, 30 mg/kg/day), 2) Arsenic acid sodium salt (3, 10, 30 mg/kg/day) and 3) Arsenic trioxide (3, 10, 30 mg/kg/day) were administered intraperitoneally (i.p.) once daily on days 0, 7 and 14.

### 2-1-C. Stock Solution

Stock solution of sodium meta-arsenite and arsenic acid sodium salt (3 mg/ml) were prepared in 5% (w/v) dextrose solution, respectively, to minimize acute arsenic toxicity. For arsenic trioxide, 9 mg of arsenic trioxide was dissolved in 1M NaOH (75 μl) followed by mixing with sterile aqua injectabilia (2175 μl) and sterile 20% (w/v) dextrose in water (500 μl). Further dilution to the desired concentration was done with a 5% dextrose solution to give a final concentration of 3 mg/ml.

### 2-1-D. Cancer Volume Measurement

The cancer volume of each animal was determined by two-dimensional measurement using calipers. The volume was calculated according to the formula: (a×b²)×0.5; wherein a and b represent two perpendicular diameters in which a (length) is the larger diameter and b (width) the smaller diameter.

Relative cancer volumes (RCV) were calculated for each individual cancer by dividing the cancer volume on day X by the cancer volume on day 0 multiplied by 100%. The cancer volume of a treatment group was expressed as the median RCV of all mice of the group. Median RCV values were used for drawing growth curves and treatment evaluation. Optimal cancer growth inhibition at a particular day within the treatment period was calculated from the median RCV values of the treated (T) versus the controlled (C) group multiplied by 100%. A T/C value<50% was considered as anti-cancer activity.

### 2-2 Results

### 2-2-A Side Effects of Sodium Meta-Arsenite and Arsenic Acid Sodium Salt

All 3 dosages of the arsenic acid sodium salt did not cause any side effects in terms of body weight and lethality demonstrated that the Maximum Tolerated Dose (MTD) was greater than 30 mg/kg/day. The T/C values of the 3 treatment groups were all greater than 74%, which indicates that the arsenic acid sodium salt did not possess anticancer activity at the dose levels studied.

At a dose level of 30 mg/kg/day, all mice of two groups that are exposed to arsenic trioxide and sodium meta-arsenite died within a few days after the first injection.

At a dose level of 10 mg/kg/day, one (1) out of four (4) mice died and lost 5% of body weight in the group of mice that is exposed to arsenic trioxide. Meanwhile, no lethality or body weight loss was observed at this dose level for the group of mice which were exposed to sodium meta-arsenite. Therefore, the MTD of both compounds was 10 mg/kg/day.

Table 5 displays that sodium meta-arsenite had anticancer activity at the ⅓ MTD and was marginal inactive at the MTD. Arsenic trioxide was active at both the MTD and ⅓ MTD. Thus, the results indicate that sodium meta-arsenite and arsenic trioxide possessed anti-cancer activity at ⅓ MTD using i.p. administration at a weekly ×3 schedule. The MTD of both compounds was less active compared with the ⅓ MTD. Arsenic acid sodium salt did not show anticancer activity at the dose levels tested.

TABLE 5
Anticancer activity of arsenic compounds in RXF 944LX cancer bearing nude mice

| | Dose | | BW loss | Drug-related | | | |
|---|---|---|---|---|---|---|---|
| Compound | mg/kg | Route & Schedule | % (day) | deaths | T/C % | SGD | Rating |
| Vehicle | 10 ml/kg | i.p., days 0, 7, 14 | +9.7 (21) | 0/4 | | | |
| Sodium | 30 | i.p., days 0, 7, 14 | NE | 4/4 | NE | NE | |
| meta-arsenite | 10 | | +8.7 (21) | 0/4 | 51 | −0.1 | − |
| | 3 | | +6.8 (21) | 0/4 | 19 | 0.4 | + |
| Arsenic acid | 30 | i.p., days 0, 7, 14 | +14 (21) | 0/4 | 88 | 0.1 | − |
| sodium salt | 10 | | +10 (21) | 0/4 | 74 | −0.2 | − |
| | 3 | | +15 (21) | 0/4 | 90 | −0.5 | − |
| Arsenic trioxide | 30 | i.p., days 0, 7, 14 | NE | 4/4 | NE | NE | |
| | 10 | | −5.0 (11) | 1/4 | 41 | −0.2 | + |
| | 3 | | +8.4 (14) | 0/4 | 31 | −0.1 | + |

BW, body weight; SGD; specific growth delay [(cancer doubling time treated minus cancer doubling control)/cancer doubling time control]; NE, not evaluable, Efficacy criteria: −T/C ≧ 50%, +T/C < 50

### 2-2-B Anticancer Activity of Sodium Meta-Arsenite and Arsenic Acid Sodium Salt on Human Renal Cancer RXF 944LX.

In the experiment with renal cancer RXF 944LX, the anticancer activity of the compounds was explored using two administration schedules; 1) intermittent schedule of 4 days×3, and 2) a daily schedule for 2 weeks (days 0-4, 7-11).

The dose levels chosen for sodium meta-arsenite and arsenic acid sodium salt in these schedules were based on the experience in the 1^{st} experiment. Additional small dose finding studies were executed.

Again, administration of the vehicle did not cause any side effects, whereas the arsenic compounds caused dose-dependent side effects.

The MTD of arsenic acid sodium salt was 45 mg/kg/day in the intermittent schedule and 16 mg/kg/day in the daily schedule.

These dosages of arsenic acid sodium salt did not cause any relevant anticancer activity in the renal cancer RXF 944LX shown in Table 6.

TABLE 6
Anticancer activity of sodium meta-arsenite and arsenic
acid sodium salt in RXF 944LX cancer bearing nude mice

| | Dose | | BW loss | Drug-related | | | |
|---|---|---|---|---|---|---|---|
| Compound | Mg/kg | Route & Schedule | % (day) | deaths | T/C % | SGD | Rating |
| Vehicle | 10 | i.p., days 0, 4, 8 | +19 (14) | 0/6 | | | |
| Arsenic acid | 45 | i.p., days 0, 4, 8 | +22 (14) | 2/6 | 100 | −0.3 | — |
| sodium salt | 30 | | +25 (14) | 1/6 | 100 | −0.2 | — |
| | 32 | i.p., days 0-4, 7, 14-18 | −10 (14) | 3/6 | NE | NE | |
| | 16 | i.p., days 0-4, 7-11 | −8 (14) | 2/6 | 76 | 0 | — |
| Sodium | 10 | i.p., days 0, 4, 8 | NE | 5/6 | NE | NE | |
| meta-arsenite | 5 | | −2 (3) | 0/6 | 84 | −0.5 | — |
| | 8 | i.p., days 0-4, 7, 14-18 | −14 (10) | 3/6 | NE | NE | — |
| | 4 | i.p., days 0-4, 7-11 | −3 (7) | 2/6 | 55 | 0.1 | — |

BW, body weight; SGD; specific growth delay [(cancer doubling time treated minus cancer doubling control)/cancer doubling time control]; NE, not valuable, Efficacy criteria: −T/C ≧ 50%, +T/C < 50

The MTD of sodium meta-arsenite in the intermittent schedule was 5 mg/kg/day in RXF 944LX. This dose did not inhibit the growth of the cancer model significantly. In the daily schedule, the MTD of sodium meta-arsenite was 4 mg/kg/day and was borderline inactive in RXF 944LX shown in Table 6.

In conclusion;

Arsenic acid sodium salt did not show anticancer activity in RXF 944LX in both administration schedules. Meanwhile, sodium meta-arsenite was borderline inactive in RXF 944LX in the daily schedule. However, sodium meta-arsenite was inactive in RXF 944LX in the intermittent schedule.

### 2-2-C Anticancer Activity of Sodium Meta-Arsenite (p.o.) Versus Arsenic Trioxide (i.p.) on Human Leukemia Models

Anticancer activity of the sodium meta-arsenite and arsenic trioxide against two human paediatric acute lymphoblastic leukemia (ALL) models in vivo, ALL-SCID 4 was investigated to compare.

ALL-SCID 4 has been characterized as a T ALL and is sensitive to six (6) out of seven (7) standard chemotherapeutic agents used in the treatment of leukemia including daunorubicin, vincristine and cyclophosphamide.

Male NOD/SCID mice (deficient in T and B lymphocytes, NK cells and functional macrophages) were transplanted s.c. on day 0 with fragments of ALL-SCID 4 cancers, which were obtained from mice used for routine passage of the cancers. Mice were randomized to the scheduled treatment groups.

Sodium meta-arsenite was administered orally at 2, 4 and 6 mg/kg/day (⅓ MTD, ⅔ MTD and MTD) once every day from day 34-54 after transplantation. The vehicle control (5% glucose in water) was administered i.p. (10 ml/kg). At the start of treatment, the mean cancer volume of the treatment groups was 178 mm³ (150-170 mm³).

Sodium meta-arsenite was dissolved in ready-to-use sterile infusion (5% (w/v) glucose in aqua injectabila). Arsenic trioxide was dissolved in 25 mM NaOH then dissolved in 5% glucose in aqua injectabila. The solutions were prepared freshly on the day of therapy for each administration. Prior to the study, small dose finding studies were carried out and demonstrated that the MTD of sodium meta-arsenite and arsenic trioxide was 6 mg/kg/day. The mice of the control group were killed on day 45 due to large cancer volume; all other mice were killed on day 55. Body weight loss and lethality were not observed in the control and sodium meta-arsenite treated groups as shown in Table 7. Significant anti-cancer activity (T/C 38%) was seen at ⅓ MTD, the higher dose was less active: T/C 55% (⅔ MTD) and 48% (MTD).

TABLE 7
Anticancer activity of sodium meta-arsenite versus arsenic
trioxide in ALL-SCID 4 cancer bearing nude mice

| | Dose | | BW loss | Drug-related | | |
|---|---|---|---|---|---|---|
| Compound | mg/kg | Route & Schedule | % (day) | deaths | T/C % | p-value |
| Vehicle | 10 ml/kg | i.p., days 34-44 | +1 (44) | 0/8 | | |
| Sodium | 2 | p.o., days 34-54 | 0 (44) | 0/8 | 38* | 0.0016 |
| meta-arsenite | 4 | | +5 (44) | 0/8 | 55* | 0.021 |
| | 6 | | +5 (44) | 0/8 | 48* | 0.016 |
| Vehicle | 10 ml/kg | i.p., days 25-35 | +5 (34) | 0/8 | | |
| Arsenic trioxide | 2 | i.p., days 25-35 | +9 (34) | 0/8 | 71 | >0.5 |
| | 4 | | +10 (34) | 0/8 | 97 | >0.5 |
| | 6 | | +10 (34) | 0/8 | 58 | >0.5 |
| Cyclophosphamide | 100 | i.p., day 25 | +4 (34) | 0/8 | 4* | 0.001 |

*Significant to control,
+significant to higher doses
Cancer doubling time ALL-SCID 4: 4.1 days for vehicle in sodium meta-arsenite experiment and 3.5 days for vehicle in As2O3 experiment Efficacy criteria: −T/C ≧ 50%, +T/C < 50

In conclusion, the side effects at high doses (MTD) were very moderate when sodium meta-arsenite was used as single agent therapy. The best efficacy was obtained at the ⅓-⅔ MTD.

### 3. Animal Toxicology

As part of the assessment of the safety profile of sodium meta-arsenite, a program of animal toxicology studies was performed to support its use in humans.

3.1 Maximum Tolerated Dose Assessment of Sodium Meta-Arsenite after Daily Oral Gavages for 14 Days in the Mouse

The objective of this oral toxicity study was to establish a MTD after repeated administration of sodium meta-arsenite to mice.

Ampoules of sodium meta-arsenite (dissolved in 10% (w/v) glucose in water) in concentrations of 1, 1.6, 2.6, 4.1 and 6.5 mg/ml were stored in a refrigerator in the dark. Based on the previous study, the following doses were selected: 10, 16, 26, and 41 and 65 mg/kg/day. Each dose was administered daily by oral gavages to groups of 4 male and 4 CD-1 mice for 14 days, followed by a 14-day recovery period.

Clinical signs were evaluated daily and body weights were recorded twice weekly. Scheduled termination was performed on day 29 (after 14 days of recovery). Macroscopic examination was performed on all animals.

The results of the study are:

10 mg/kg/day; No findings were noted.

16 mg/kg/day; Piloerection on the head was noted for one male on days 4 and 5.

26 mg/kg/day; No unscheduled mortality occurred. A swollen abdomen (day 2 of treatment to day 3 of recovery), piloerection (day 2 to 6 of treatment) and hunched posture (day 2 of treatment) were observed in the majority of males. Hunched posture (day 2 to 11) and a swollen abdomen (day 3 of treatment to day 3 of recovery) were noted in one female. Salivation was recorded on day 2 of treatment for one female.

41 mg/kg/day; All males died on days 2-4 and all females on days 2-7. Prior to death, a swollen abdomen, lethargy, flat/hunched posture, piloerection, diarrhoea, hypothermia and/or a pale appearance were recorded. Body weight loss (males) or low body weight gain (females) was noted in surviving animals on day 4 of treatment. At necropsy, discoloration and crateriform retractions were noted in the stomach of in both sexes.

65 mg/kg/day; All animals died on days 2 or 3, all females died on day 1 or 2. Prior to death, a swollen abdomen, lethargy, flat/hunched posture, slow breathing, piloerection, diarrhea, a pale appearance and/or hypothermia were recorded. At necropsy, discoloration of the stomach was noted in both sexes; in addition emaciation was noted in one female.

### Conclusion

The MTD (or LD₁₀ value) of sodium meta-arsenite in mice was calculated to be 32 mg/kg/day for males and females. Due to the mortality distribution, no 95% confidence interval could be determined for both males and females.

3.2 Maximum Tolerated Dose Assessment of Sodium Meta-Arsenite after Daily Oral Gavages for 14 Days in the Rat

The objective of this oral toxicity study was to establish a MTD after repeated administration of sodium meta-arsenite to rats.

Ampoules of sodium meta-arsenite (dissolved in 10% (w/v) glucose in water) in concentrations of 1.2, 2.2, 3.8, 7.0 and 12.6 mg/ml were stored in a refrigerator in the dark. Based on the previous study, the following doses were selected: 6, 11, 19, and 35 and 63 mg/kg/day. Each dose was administered daily by oral gavages to groups of 4 male and 4 female Wistar rats for 14 days, followed by a 14-day recovery period.

Clinical signs were evaluated daily, and body weights were recorded twice weekly. Scheduled termination was performed on day 29 (after 14 days of recovery). Macroscopic examination was performed on all animals.

The results of the study are:

6 mg/kg/day; No findings were noted.

11 mg/kg/day; Hunched posture was noted in one male from day 7-13 of treatment, diarrhea was observed for one male on day 6.

19 mg/kg/day; Hunched posture (day 6-14 of treatment) was noted in 2 males; in addition a swollen abdomen (day 6 to 8 of treatment), diarrhea (day 7 of treatment) and salivation (day 13 of treatment) were noted in one male. Hunched posture (day 6-11) and salivation (day 13-14 of treatment) were noted in one female. Slightly low body weight was found for males during the 2nd week of treatment. At necropsy, foci were found in the forestomach in the majority of the animals.

35 mg/kg/day; One male was found dead on day 13 of treatment, all females died between days 3 to 6. In males a swollen abdomen (day 3 of treatment to day 3 of recovery), piloerection (day 9 of treatment to day 7 of recovery), hunched posture (day 6 of treatment to day 6 of recovery), diarrhea (day 2 to 4 of treatment), lethargy (day 11 of treatment to day 1 of recovery), emaciation (day 13 of treatment to day 7 of recovery) and salivation (day 8 of treatment to day 2 of recovery) were noted. For females a swollen abdomen, lethargy, hunched posture, piloerection, diarrhea, emaciation, ptosis, hypothermia and/or a pale appearance were recorded prior to death.

Low body weights and body weight gains were noted during treatment in males, body weights improved during recovery. At necropsy, findings comprised abnormalities in the forestomach in both sexes, foci in the liver of 2 males and an accentuated lobular pattern of the liver in one male.

63 mg/kg/day; All animals died on days 2 to 5. Prior to death, a swollen abdomen, lethargy, hunched posture, ptosis, piloerection, diarrhea, a pale appearance and/or hypothermia were recorded. Slight body weight loss was noted in surviving animals on day 3 of treatment. Discoloration of the forestomach was noted in the majority of animals, in addition discoloration of the ileum was recorded for one male.

### Conclusion

The MTD (or LD₁₀ value) of sodium meta-arsenite in rats was calculated to be 33 mg/kg/day for males and 25 mg/kg/day for females. Due to the mortality distribution, no 95% confidence interval could be determined for both males and females.

In conclusion, the MTD of sodium meta-arsenite administered orally once daily for 14 consecutive days is in the mouse 32 mg/kg/day or 96 mg/m²/day. This means that the starting dose for a phase I clinical study is 0.1×MTD=9.6 mg/m²/day in the mouse or 1/12×9.6 mg/m²/day=0.8 mg/m²/day in human or 1.5 mg/day.

The MTD of the mouse (32 mg/kg/day for both sexes) and the rat (33 mg/kg/day for males and 25 mg/kg/day for females) are similar. This is a rare finding in toxicology studies with anticancer drugs. Usually the rat MTD is a factor two smaller. This concurs with the fact that the rat is different than other mammals in that it accumulates arsenic in the erythrocytes after receiving inorganic arsenic orally or parentally. This implies that the rat is not a good model for further toxicological studies with arsenic compounds and sodium meta-arsenite in particular. Therefore, the main toxicological study was only performed in mice. Furthermore, a MTD and kinetics study was carried out in the pig as the second species.

3.3 Maximum Tolerated Dose Assessment and Kinetic Analysis of Sodium Meta-Arsenite after Daily Oral Administration for 14 Days in the Pig.

Since the rat was not suitable as the second species, pigs were selected as the second species, having assumed kinetics of sodium meta-arsenite comparable to humans.

The aims of this study were to establish a MTD and the kinetics of sodium meta-arsenite after repeated administration to pigs.

Sodium meta-arsenite was formulated using enteric-coated capsules (size no. 2) for a toxicology study in pigs. Cellulose-based clear capsules contained 1) sodium meta-arsenite as an active ingredient, 2) microcrystalline cellulose and colloidal silicon dioxide as excipients. The coating material of the capsules comprised of Eudragit L 12.5 (acrylic polymer) and Macrogol 6000 (polyethylene glycol as softener). It is resistant to gastric fluid at least for 1 hour.

The oral route was selected, as it is the intended route of administration in human. Based on previous studies, the following doses were selected: 7.5 and 10 mg/pig (equivalent to 24.5 and 32.5 mg/man). Capsules (enteric coated, size no. 2) of sodium meta-arsenite in concentrations of 7.5 and 10 mg/capsule, formulations, were stored in a refrigerator in the dark. Each dose was administered orally by gavages to groups of 12 male growing pigs (large white×landrace×pietrain) for 14 days, followed by a 14-day recovery period. The mean weight of the pigs was 16.8±2.3 kg at the beginning of the study.

Mortality and viability were checked twice daily, clinical examinations were performed daily and body weight was recorded twice a week. Scheduled termination was performed on day 29 (after 14 days of recovery). Macroscopic examination was performed on all animals and samples from brain (cerebellum, mid brain, cortex), heart, kidney, liver, lung, spleen, stomach wall, small intestine and large intestine taken for histopathology.

Blood samples (10 ml) of both dose groups were collected for hematology and biochemistry analysis on days −2, −1, 0, 7, 14, 21, 28. The samples collected during the treatment period were taken prior to the administration of the next drug.

For the determination of the pharmacokinetics blood samples were collected on the day before treatment (day 0) and days 7 and 14 of the dosing schedule at 0, 5, 10, 30, 60, 120, 180 and 360 minutes after the oral administration of sodium meta-arsenite. On day 28 only one sample was taken to determine the remaining plasma level of sodium meta-arsenite. Blood samples were centrifuged after collection and plasma and the remaining blood cell samples were stored at −20° C.

No pigs died or were killed in extremis during the treatment and recovery period. During the duration of 2-week treatment period no clinical signs of intolerance were observed. Weight gains were similar in all four groups during the whole study period and were consistent with those of growing pigs of this age.

No (statistical) significant differences were observed in hematological and biochemical parameters.

Macroscopical and microscopical lesions observed post mortem were considered minor and of little or no pathological significance. No dose effect was seen on either the severity or the frequency of the abnormal findings post mortem.

No dose related effect was demonstrated in this study. In absence of an untreated control group, the absence of any effect cannot clearly stated, but it must be noted that all pigs remained healthy throughout the trial with hematological and biochemical parameters remaining normal.

Thus, sodium meta-arsenate did not induce any drug related side effects in pigs at the doses of 7.5 and 10 mg/day.

3.4 Subacute Toxicity Study of Sodium Meta-Arsenite after Daily Oral Gavages for 14 Days in the Mouse Followed by a 28-Day Recovery Period

The nature and purpose of this subacute oral toxicity study was to determine the extent to which cumulative toxicity occurred with repeated administration of sodium meta-arsenite for 14 days to mice. Moreover, the study was intended to define the most likely target organs for toxicity and to investigate the reversibility of toxicity.

Ampoules of sodium meta-arsenite (dissolved in 10% (w/v) glucose in water) in concentrations of 0, 1.6 and 3.2 mg/ml were stored in a refrigerator in the dark. Based on the previous study, the following doses were selected: 0, 16 (½ MTD) and 32 mg/kg/day (MTD). Each dose was administered daily by oral gavages to groups of 20 CD-1 male mice for 14 days, followed by a 28-day recovery period.

Ten animals of each group were killed on day 15 (main groups). The remaining animals (recovery groups) were killed on day 43 after a recovery period of 28 days. Blood samples were collected from all main group animals on day 5 to determine effects on the hematology. Blood samples were also collected at the end of the treatment period (day 15, all main group animals) and after recovery (day 43, all recovery group animals) for hematology and clinical biochemistry.

Clinical signs were evaluated daily and body weights were recorded twice weekly. Macroscopic examination was performed at termination. Organ weight measurements were performed at scheduled termination, i.e. day 15 (main groups) and day 43 (recovery groups). Microscopic examination was performed on a selection of tissues of both main and recovery animals.

The results are summarized as follows:

16 mg/kg; Clinical signs consisted mainly of swelling of the abdomen and piloerection. Incidentally lethargy was noted.

Affected erythrocyte parameters (lower erythrocyte count, hemoglobin and haematocrit) were noted at day 5 and the end of treatment. At the end of treatment lower leucocyte counts, increased reticulocyte counts and an increased red cell distribution width were noted.

After necropsy, crateriform retractions of the fore stomach were noted at the end of treatment.

Microscopic examination revealed atrophy of the lymphoid organs (Peyer's patches, thymus), ulceration and hyperplasia of the fore stomach epithelium, necrosis of liver and gall bladder and myeloid hyperplasia and congestion of the sternal bone marrow. After recovery, myeloid hyperplasia of the sternal bone marrow was present.

32 mg/kg; Premature mortality occurred during the treatment phase in 7 males. Prior to death several clinical signs were noted in these animals. Causes of death were considered to be fore stomach ulceration in 5/7 and not evident in 2/7 animals.

Clinical signs consisted mainly of swelling of the abdomen and piloerection. Incidentally lethargy, pale or lean appearance or hunched posture was noted.

Lower leucocyte count, platelet count, affected erythrocyte parameters (lower erythrocyte count, hemoglobin and haematocrit) and low reticulocyte counts were noted on day 5 and at the end of treatment. After recovery, increased reticulocyte counts were recorded.

Decreased glucose, total protein and albumin concentrations and increased sodium, calcium and inorganic phosphorus concentrations were recorded only at the end of treatment.

At necropsy, crateriform retractions of the forestomach and a reduced thymus size were noted after treatment. After recovery, crateriform retractions were noted in one animal.

Decreased absolute and relative spleen and thymus weight and increased relative liver weight were noted at the end of treatment. At the end of the recovery period relative liver weights remained slightly higher.

Microscopic examination revealed atrophy of the lymphoid organs (mesenteric and mandibular lymph nodes, Peyer's patches, spleen and thymus), ulceration and hyperplasia of the fore stomach epithelium, ulceration of the caecum, necrosis of the liver and gall bladder, hypocellularity, myeloid hyperplasia and congestion of the sternal bone marrow, acute cortical tubular necrosis of the kidney and a haemorrhage in the mandibular lymph nodes.

After recovery, microscopic examination revealed atrophy of the Peyer's patches, hyperplasia of the fore stomach epithelium, necrosis of the gall bladder, acute cortical tubular necrosis of the kidney, myeloid hyperplasia of the sternal bone marrow.

Incidences and severities of the findings were generally reduced at the lower level. At both levels incidences and severities were generally reduced following a 28-day treatment free recovery period, indicating partial reversibility of the induced changes.

The crateriform retractions observed at necropsy in both treatment groups correlate with the microscopic findings, ulceration and hyperplasia of the forestomach epithelium. The relevance of this observation for human risk assessment is doubted, as the forestomach is a species-specific tissue, which men lack.

In addition, necrosis of the gall bladder and/or liver was recorded at microscopic examination at the end of treatment in several animals of both treatment groups. After recovery, necrosis of the gall bladder was present in one animal treated at 32 mg/kg/day. Incidentally, treatment of 32 mg/kg/day resulted in acute cortical tubular necrosis of the kidney. This was recorded in one animal at the end of treatment and one animal after recovery. No further corroborative findings or mechanism of action was found for these alterations but a relation to treatment can not be excluded.

After recovery, findings in these target organs were either completely resolved or were noted at a lower incidence and severity (forestomach, bone marrow, gall bladder, kidney Peyer's patches).

### Conclusion

Based on the results, the main target organs for toxicity are bone marrow, lymphoid organs and the forestomach; in addition gall bladder, liver and kidney were considered as target organs. Effects in these organs were noted in both dose groups, most pronounced in the highest dose group. The observed changes in the blood forming organs are considered to be consistent with the pharmacological action of sodium meta-arsenite as an anticancer drug.

Thus, sodium meta-arsenite, administered orally once daily for 14 consecutive days, induced in the mouse dose-dependent side effects that had either completely disappeared or were found at a lower incidence and severity after recovery. The main target organs are bone marrow and lymphoid organs.

Disclosures in the current application do not limit the scope of invention. Effect of Sodium meta-arsenite to other cancer cells such as ovarian cancer, mammary cancer, melanoma, prostate cancer, etc. according to requirements by FDA or EORTC or other country's guidelines for approval for new anti-cancer drugs are induced.

What is claimed is:

1. A method of curing human cancer with sodium meta-arsenite (AsO₂⁻) in a starting dose calculated as 1.5 mg/day, which is calculated from a series of experiments, that are required by FDA/EORTC and other country's guidelines for approval for new anti-cancer drugs, comprising;
an in vitro cytotoxic activity test with
six kinds of human cancers of colon cancer, leukemia, non-small cell lung cancer, melanoma, renal cancer, and uterine body cancer with nine cancer cell lines of;
HT29 from colon cancer,
CCRF-CEM, K562 from leukemia, and U937 from lymphoma,
LXFL 529L from non-small cell lung cancer,
MEXF 462NL and MEXF 514L from melanoma,
RXF 944L from renal cancer, and
UXF 1138 from uterine body cancer,
and
eight kinds of human cancers of colon cancer, gastric cancer, lung cancer, mammary cancer, melanoma, ovarian cancer, prostate cancer, and renal cancer with twelve cancer cell lines of;
DLD1 from colon cancer,
GXF 251L from gastric cancer,
LXFA 629L and LXFE 66NL from lung cancer,
MAXF 401NL from mammary cancer,
MEXF 276L from melanoma,
OVCAR3 and OVXF 899L from ovarian cancer,
DU145 and PC3 from prostate cancer, and
RXF 93NL and RXF 486L from renal cancer,
and
eleven kinds of human cancers of colon cancer, gastric cancer, leukemia, lymphoma, lung cancer, mammary cancer, melanoma, ovarian cancer, prostate cancer, renal cancer, and uterine body cancer with forty-one cancer cell lines of,
DLD1, HCC2998, HCT116, HT29, SW620, and CXF 94L from colon cancer,
GXF 251L from gastric cancer,
LECL 226, CCRF-CEM, HL60, MOLT4 from leukemia,
H460, LXFA 289L, LXFA 526L, LXFA 629L, LXFE 66NL, and LXFL 529L from lung cancer,
MCF7, MDA231, MDA468, and MAXF 401NL from mammary cancer,
HT144, MALME3M, SKMEL2, SKMEL28, MEXF 276L, MEXF 462NL, and MEXF 514L from melanoma,
A2780, IGROVE1, OVCAR3, SKOV3, and OVXF 899L from ovarian cancer,
22RV1, DU145, PC3, and PC3M from prostate cancer,
RXF 393NL, RXF 86L, and RXF 944L from renal cancer, and
UXF 1138L from uterine body cancer
at a concentration of 100 μg of sodium meta-arsenite/ml solution of dimethyl sulfoxide mixed with sodium meta-arsenite (AsO₂⁻) and diluted with culture medium RPMI 1640,
and
an in vivo anticancer activity against nude mice, which were killed before the cancers reached mean diameters of approximately 16 mm according to the Animal Experimentation Rules, with
human renal cancer cell line RXF 944LX, which were obtained from xenografts in serial passage in nude mice and implanted subcutaneously (s.c.) in a flank of male NRMI nude mice, by measuring the volume of the cancer cell with a formula of (a×b²)×0.5, wherein a and b represent two perpendicular diameters in which a (length) is the larger diameter and b (width) the smaller diameter and measuring body weight of the mouse twice per week while injecting 10 mg/kg/day of sodium meta-arsenite (AsO₂⁻) every week for two weeks from day 0.
and
Leukemia cell, which were obtained from xenografts in serial passage in male NOD/SCID mice which were transplanted s.c. on day 0 of ALL-SCID4 cancers, by measuring the volume of the cancer cell with a formula of (a×b²)×0.5, wherein a and b represent two perpendicular diameters in which a (length) is the larger diameter and b (width) the smaller diameter, and measuring body weight of the mouse once every day from day 34 after transplantation while orally administering 2 mg/kg/day of sodium meta-arsenite (AsO₂⁻) every week for two weeks from day 0.
and
maximum tolerated oral dose assessment
in mouse for 32 mg/kg/day for both sexes after daily oral gavages for 14 days followed by a 14-day recovery period,
and
in rats for 33 mg/kg/day for males and 25 mg/kg/day for females after daily oral gavages for 14 days followed by a 14-day recovery period,
and
sodium meta-arsenite (AsO₂⁻) did not induce any side effects in pigs at the doses of 7.5 mg/day/20 kg pig and 10 mg/day/20 kg pig after daily oral gavages for 14 days followed by a 14-day recovery period
and
subacute toxicity study after daily oral gavages for 14 days in the mouse followed by a 28-day recovery period confirming complete resolving of side effects in target organ that was affected by sodium meta-arsenite (AsO₂⁻) during the 14 days of oral gavages.

2. A method of curing human cancer with sodium meta-arsenite (AsO₂⁻) in a starting dose calculated as 1.5 mg/day of claim 1, wherein the human cancer is Renal Cancer.

3. A method of curing human cancer with sodium meta-arsenite (AsO₂⁻) in a starting dose calculated as 1.5 mg/day of claim 1, wherein the human cancer is Leukemia.

4. A method of curing human Renal Cancer with sodium meta-arsenite (AsO₂⁻) in a dose of 20 mg/day.

5. A method of curing human Renal Cancer with sodium meta-arsenite (AsO₂⁻) in a dose of 0.1 mg/day.

6. A method of curing Leukemia with sodium meta-arsenite (AsO₂⁻) in a dose of 20 mg/day.

7. A method of curing Leukemia with sodium meta-arsenite (AsO₂⁻) in a dose of 0.1 mg/day.

8. A method of curing human Renal Cancer with sodium meta-arsenite (AsO₂⁻).

9. A method of curing Leukemia with sodium meta-arsenite (AsO₂⁻).
